(19) United States
(12) Patent Application Publication
(10) Pub. No.: US 2010/0312336 A1
(43) Pub. Date: Dec. 9, 2010

(54) ZONAL DIFFRACTIVE MULTIFOCAL INTRAOCULAR LENS WITH CENTRAL MONOFOCAL DIFFRACTIVE REGION

(76) Inventors: Xin Hong, Fort Worth, TX (US); Xiaoxiao Zhang, Fort Worth, TX (US)

Correspondence Address: ALCON, IP LEGAL, TB4-8, 6201 SOUTH FREEWAY, FORT WORTH, TX, 76134, US

(21) Appl. No.: 12/780,244

(22) Filed: May 14, 2010

Related U.S. Application Data
(60) Provisional application No. 61/185,512, filed on Jun. 9, 2009.

(51) Int. Cl. A61F 2/16; G02C 7/06

(52) U.S. Cl. 623/6.27; 351/177; 351/168

(57) ABSTRACT

An ophthalmic lens includes an optic having an anterior surface and a posterior surface. The lens also includes a monofocal diffractive structure disposed on one of said surfaces for providing a diffractive focusing power. The lens further includes at least one multifocal diffractive structure disposed on one of said surfaces for providing a plurality of diffractive focusing powers. The multifocal diffractive structure is adapted to provide chromatic aberration compensation for near vision.

### PRIORITY APPLICATIONS

This application claims priority to U.S. provisional application Ser. No. 61/185,512, filed on Jun. 9, 2009, the contents which are incorporated herein by reference.

### RELATED APPLICATIONS

This application is related to co-pending application Ser. No. ______, entitled “IOL WITH VARYING CORRECTION OF CHROMATIC ABERRATION” claiming priority to application Ser. No. 61/185,510 filed on the same day as the application to which the present application claims priority.

### BACKGROUND

The present invention relates generally to multifocal ophthalmic lenses, and more particularly to multifocal intraocular lenses (IOLs) that provide compensation for chromatic aberrations.

Intraocular lenses are employed routinely to replace an occluded natural crystalline lens via cataract surgery. In other cases, an intraocular lens can be implanted in a patient's eye while retaining the natural crystalline lens to improve the patient's vision. Both monofocal and multifocal IOLs are known. While monofocal IOLs provide a single focusing power, multifocal IOLs can provide multiple focusing powers—typically two—to provide a degree of accommodation, commonly known as pseudo accommodation.

Many conventional IOLs, however, exhibit chromatic aberrations that can degrade their efficiency in concentrating the light energy incident thereon onto the patient's retina. Nor are such conventional IOLs typically designed to address the chromatic aberrations inherently present in the optical system of the patient's eye. In addition, many conventional multifocal IOLs may not be optimal for distance viewing as they direct a significant portion of the light energy to a near focus even for small pupil sizes.

Accordingly, there is a need for enhanced ophthalmic lenses, and particularly improved IOLs, that address the above shortcomings of conventional IOLs.

### SUMMARY

In particular embodiments of the present invention, an ophthalmic lens includes an optic having an anterior surface and a posterior surface. The lens also includes a monofocal diffractive structure disposed on one of said surfaces for providing a diffractive focusing power. The lens further includes at least one multifocal diffractive structure disposed on one of said surfaces for providing a plurality of diffractive focusing powers. The multifocal diffractive structure is adapted to provide chromatic aberration compensation for near vision.

In other embodiments, a method for manufacturing an ophthalmic lens includes determining a first surface profile for a monofocal diffractive structure disposed on either an anterior surface or a posterior surface of an IOL for providing a diffractive focusing power. The method further includes determining a second profile for at least one multifocal diffractive structure disposed on either the anterior surface or the posterior surface of the IOL for providing a plurality of diffractive focusing powers. The multifocal diffractive structure is adapted to provide chromatic aberration compensation for near vision. The method further includes manufacturing the IOL.

In many embodiments, the present invention provides ophthalmic lenses (e.g., IOLs) that employ a monofocal diffractive structure as well as a bifocal diffractive structure to provide enhanced distance and near vision. By way of example, in some cases, a monofocal diffractive structure disposed on a central region of one of the lens surfaces can provide a single far-focus optical power, which can be selected to be substantially equal to a refractive far-focus optical power provided by the lens due to the base profiles of its optical surfaces. While the refractive focusing power would exhibit a positive longitudinal chromatic aberration, the monofocal diffractive structure would exhibit a negative longitudinal chromatic aberration that can counteract the positive chromatic aberration so as to direct more light energy to the lens's far focus. In case of IOLs, the negative chromatic aberration of the monofocal diffractive structure can also counteract the inherent positive chromatic aberration of the patient's eye to provide better far vision. The bifocal diffractive structure, which in many embodiments is disposed on an annular region surrounding the monofocal diffractive structure, provides a distance as well as a near optical power. Similar to the monofocal diffractive structure, the bifocal structure exhibits a negative longitudinal chromatic aberration that can, e.g., counteract the eye's positive chromatic aberration for near vision.

The use of a monofocal diffractive structure as well as a bifocal diffractive structure can provide a patient with pseudoaccommodation while directing the light energy primarily to the far focus for small pupil sizes (the monofocal structure provides primarily a single focusing power). In other words, in many embodiments, the distribution of light energy directed to the far and near foci of the lens changes as a function of pupil size such that at small pupil sizes the light energy is primarily directed to the far focus. As the pupil size increases beyond the diameter of the monofocal diffractive structure, the bifocal diffractive structure directs some of the light energy to its near focus. In many cases, the bifocal structure is surrounded by a refractive surface portion, which contributes to the far-focus optical power as the pupil size increases further such that a portion of the incoming light is incident on the refractive surface portion.

In another aspect, the present invention provides an ophthalmic lens (e.g., an intraocular lens (IOL)), which comprises an optic having an anterior surface and a posterior surface. A monofocal diffractive structure is disposed on one of those surfaces for providing a single diffractive focusing power, and at least one multifocal diffractive structure is disposed on one of those surfaces for providing a plurality of diffractive focusing powers.

In certain embodiments, the monofocal diffractive structure can provide a focusing power that corresponds to a far-focus optical power of the lens. The multifocal diffractive structure, in turn, can contribute to the lens's far-focus optical power and also generate a near-focus optical power.

By way of example, the monofocal diffractive structure can be disposed on a central region of the lens's anterior surface while the multifocal diffractive structure can be in the form of an annular region surrounding the monofocal diffractive structure. While in some implementations the multifocal diffractive structure extends from an outer boundary of the monofocal structure to the periphery of the optic, in other embodiments the multifocal structure is truncated such that the surface on which it is disposed includes an outer refractive region. In other cases, a refractive surface region can separate the monofocal diffractive structure from the multifocal structure.

In a related aspect, the monofocal and the multifocal diffractive structures can be formed by a plurality of diffractive echelettes that are separated from one another by a plurality of steps. In some embodiments, the step heights associated with the monofocal and/or the multifocal diffractive structures are apodized, e.g., the step heights decrease as a function of increasing distance from a center of the lens.

By way of example, in some cases in which a monofocal structure is surrounded by an adjacent annular bifocal structure, the height of the step that separates the central diffractive zone of the monofocal structure from an adjacent outer zone can correspond to one wavelength (λ) at a design wavelength (e.g., 550 nm) with the subsequent steps exhibiting a decrease in height such that the step separating the monofocal diffractive structure from the bifocal structure would exhibit a height corresponding to one-half wavelength (λ/2) at the design wavelength. The subsequent steps associated with the bifocal structure can also exhibit decreasing heights so as to provide a smooth transition between the bifocal structure and a refractive outer region of the surface.

In other cases, the step heights associated with the monofocal and/or the multifocal diffractive structures can be substantially uniform (e.g., about 1λ for the monofocal structure and about λ/2 for the multifocal structure).

In another aspect, an ophthalmic lens (e.g., an IOL) is disclosed that includes an optic having an anterior surface and a posterior surface. A monofocal diffractive region is disposed on a central portion of one of those surfaces, and a bifocal diffractive annular region surrounds the monofocal diffractive region. The monofocal diffractive region can provide a far-focus optical power and the bifocal diffractive annular region can provide a far-focus as well as a near-focus optical power.

In another aspect, the invention provides an ophthalmic lens that includes an optic having an anterior surface and a posterior surface. A monofocal diffractive structure is disposed on one of those surfaces so as to provide a far-focus optical power. The monofocal diffractive structure can provide a negative longitudinal chromatic aberration that can compensate for the positive chromatic aberration associated with the refractive focusing power of the lens and/or that of the eye to provide, e.g., enhanced far vision. A bifocal diffractive structure is disposed on one of the surfaces (e.g., on the surface on which the monofocal structure is disposed) so as to provide a far-focus as well as a near-focus optical power.

In a related aspect, in the above ophthalmic lens, the bifocal diffractive structure can exhibit a negative longitudinal chromatic aberration, which can, e.g., compensate for the positive chromatic aberration of the eye for near vision.

In another aspect, an intraocular lens is disclosed that includes an optic having an anterior surface and a posterior surface. A monofocal diffractive structure is disposed on a portion of those surfaces, e.g., a central region of the anterior surface, and a multifocal diffractive structure (e.g., a bifocal diffractive structure) is disposed on an annular region of those surfaces so as to surround the monofocal diffractive structure. The base profile of the anterior and/or the posterior surface exhibits a selected degree of asphericity (e.g., it exhibits progressively larger deviations from a spherical profile as a function of increasing distance from the center of the lens) so as to ameliorate, and preferably eliminate, spherical aberration effects. In some cases, the asphericity can be characterized by a conic constant in a range of about −1030 to about −11.

In another aspect, a method for correcting vision is disclosed that includes providing an IOL for implantation in an eye of a patient, where the IOL includes an optic comprising a monofocal diffractive structure disposed on an optical surface thereof as well as a multifocal diffractive structure disposed on the same or another optical surface of the lens. The IOL can be implanted in an eye of a patient, e.g., to replace an occluded natural lens or to augment the patient's natural lens.

Further understanding of various aspects of the invention can be obtained by reference to the following detailed description in conjunction with the drawings, which are discussed briefly below.

### BRIEF DESCRIPTION OF THE DRAWINGS

FIG. 1A is a schematic side view of an IOL in accordance with an embodiment of the invention,

FIG. 1B shows a profile of the anterior surface of the IOL depicted in FIG. 1A from which the base profile of the anterior surface has been subtracted,

FIG. 2 is a schematic side view of an IOL with a diffractive structure having uniform step heights according to another embodiment of the invention,

FIG. 3 is a schematic side view of an IOL having a multifocal diffractive structure extending to a periphery of the IOL according to another embodiment of the invention,

FIG. 4 is a schematic side view of an IOL having an annular refractive region separating first and second diffractive structures according to another embodiment of the invention,

FIG. 5 is a schematic side view of an IOL in accordance with another embodiment of the invention in which the posterior surface of the lens exhibits an aspheric base profile for controlling spherical aberrations effects, and

FIG. 6 is a flowchart illustrating a method of manufacturing an IOL according to a particular embodiment of the present invention.

### DETAILED DESCRIPTION

The present invention generally provides multifocal ophthalmic lenses, e.g., multifocal intraocular lenses (IOLs), that employ a monofocal diffractive structure to provide primarily a single focusing power (e.g., a far-focus optical power) and a multifocal diffractive structure (typically a bifocal diffractive structure) to provide a plurality of focusing powers (e.g., a far-focus as well as a near-focus optical power). In the embodiments that follow, the salient features of various aspects of the invention are discussed in connection with intraocular lenses (IOLs). The teachings of the invention can also be applied to other ophthalmic lenses, such as contact lenses. The term “intraocular lens” and its abbreviation “IOL” are used herein interchangeably to describe lenses that are implanted into the interior of the eye to either replace the eye's natural lens or to otherwise augment vision regardless of whether or not the natural lens is removed. Intracorneal lenses and phakic intraocular lenses are examples of lenses that may be implanted into the eye without removal of the natural lens.

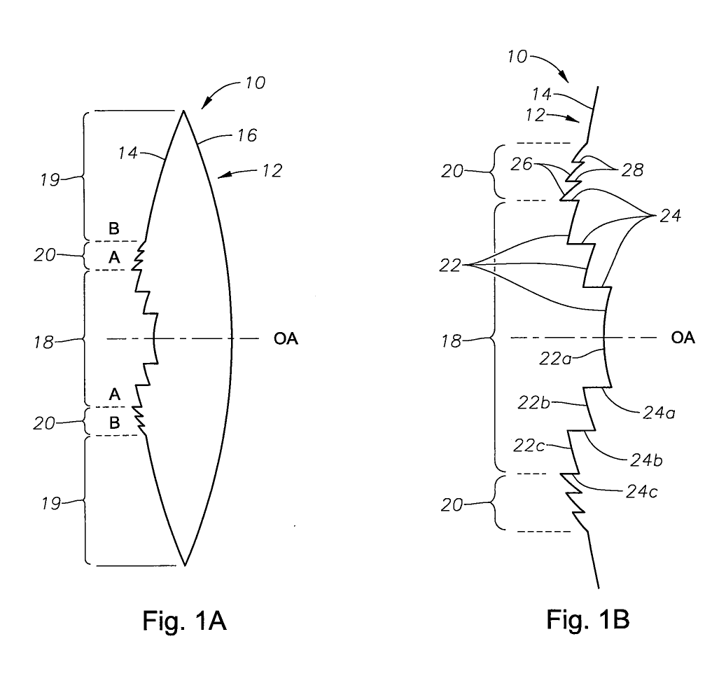

FIGS. 1A and 1B schematically depict a multifocal intraocular lens (IOL) 10 in accordance with one embodiment of the invention that includes an optic 12 having an anterior surface 14 and a posterior surface 16 disposed about an optical axis OA. A monofocal diffractive structure 18 is disposed on a central portion of the anterior surface, and is surrounded by a bifocal diffractive structure 20, which extends from an outer boundary (A) of the monofocal structure 18 to an inner boundary (B) of an outer refractive region 19 of the anterior surface. As discussed in more detail below, the monofocal diffractive structure 18 provides a single diffractive focusing power while the bifocal diffractive structure 20 primarily provides two diffractive focusing powers. More specifically, in this example, the monofocal diffractive structure provides a far-focus optical power, e.g., one in a range of about −5 to about +55 Diopters (D) and more typically in a range of about 6 to about 34 D, or in a range of about 18 to about 26 D. The bifocal diffractive structure, in turn, provides a far-focus optical power as well as a near-focus optical power. In many implementations, the near-focus optical power can be in a range of about 1 to about 4 Diopters (D), and more typically in a range of about 2 to about 3 D. In this exemplary embodiment, the far-focus power of the bifocal structure is substantially equal to the optical power provided by the monofocal diffractive structure. In other cases, the far-focus optical power of the diffractive structure can be different (e.g., by a value in a range of about 0.25 D to about 2 D, and preferably in a range of about 0.5 D to about 1 D) from the optical power of the monofocal structure, e.g., to enhance depth-of-field for distance vision.

As shown in FIG. 1A, in this embodiment both the anterior surface 14 and the posterior surface 16 of the IOL 10 have generally convex base profiles. In this example, the curvatures of the base profiles of the anterior and posterior surfaces are such that the lens body contributes refractively to the IOL's far-focus optical power. Further, as noted above, an outer refractive region 19 of the anterior surface extends from the outer boundary of the bifocal diffractive structure to the periphery of the lens, and contributes refractively to the lens's far-focus optical power for large pupil sizes, e.g., in low light conditions.

Alternatively, the curvatures of the anterior and the posterior surfaces can be selected such that the lens body would contribute refractively to the lens's near-focus optical power. In other cases, the anterior and posterior surfaces can have substantially flat profiles such that the near and far-focus optical power of the lens are due to the diffractive contributions from the monofocal and bifocal diffractive structures with no substantial (if any) refractive contribution from the lens body.

The optic can be formed of any suitable biocompatible material, including a plurality of biocompatible polymeric materials. Some examples of such materials include, without limitation, a soft acrylic material utilized for forming commercial lenses commonly known as Acrysof (a cross-linked copolymer of 2-phenylethyl acrylate and 2-phenylethyl methacrylate), silicone and hydrogel. Though not shown, the IOL 10 can also include a plurality of fixation members (e.g., haptics) that can facilitate its placement in a patient's eye.

With reference to FIG. 1B, the monofocal diffractive structure 18 includes a plurality of diffractive echelettes 22 separated from one another by a plurality of step heights 24 such that the diffractive structure 18 diffracts light into a single order (m), which is in this case the first order. In this example, the step heights 24 exhibit decreasing heights as a function of increasing distance from the center of the anterior surface (i.e., the intersection of the optical axis with the base curve of the anterior surface). In particular, in this case, the step 24a separating the centermost diffractive echelette 22a from the second diffractive echelette 22b corresponds to a phase shift of about 2π (2 pi) for a selected design wavelength (e.g., 550 nm) with the step heights decreasing to a value corresponding to a phase shift of about π (pi) for the step height 24c, which separates the monofocal diffractive structure from the bifocal diffractive structure. In this manner, a smooth transition between the monofocal and the bifocal diffractive structures can be achieved. Alternatively, the shift between π to 2π can be accomplished by changing the radial spacing between echelettes while maintaining the step height relationship between consecutive echelettes or by some combination of changing step heights and radial spacing between echelettes.

In this embodiment, the radial locations of the diffractive zones of the monofocal diffractive structure can be defined in accordance with the following relation:

rₘ²=mλf_{power} (1)

In this example, the profile of each echelette 22 is a fragment of a hyperboloid of revolution. The distance between the highest and the lowest point of an echelette (zₘₐₓ) is substantially uniform across the zones. A design parameter of the lens (α) can be adjusted to direct light to a desired order of the lens with the other orders receiving negligible contributions. More particularly, the parameter (α) can be defined in accordance with the following relation:

$$α = ( {n}_{p} - {n}_{e} ) \frac{{z}_{max}}{{λ}_{0}} \qquad ( 2 )$$

wherein nₚ denotes the index of refraction of the material from which the lens is formed, nₑ is the index of refraction of the medium surrounding the lens, and λ₀ denotes the wavelength of the incident light in vacuum.

In this example, the design parameter (α) is set to 1 (one) in order to cause the diffractive structure to diffractively direct the light incident thereon to its first order diffraction focus. Hence, the diffractive structure 18 functions as a monofocal lens that diffractively directs the light incident thereon (taking into account scattering and some leakage to other orders) onto a single focus corresponding to its first diffraction order. As noted above, in this example, the IOL's monofocal diffractive focus corresponds to IOL's far focus, though in other embodiments it can correspond to its near focus.

With reference to FIG. 1B, the bifocal diffractive structure 20 is also formed of a plurality of diffractive echelettes 26 that are separated from one another by a plurality of steps 28. However, the diffractive echelettes 26 and the steps 28 are configured such that the diffractive structure 20 provides primarily two foci: a far-focus and near-focus. In this example, the far-focus power of the bifocal structure 20 is substantially equal to the monofocal optical power of the monofocal diffractive structure 18.

In this exemplary implementation, the steps separating different echelettes of the bifocal diffractive structure exhibit decreasing heights as a function of increasing radial distance from the center of the anterior surface 14 such that the step height reaches a vanishing value at the boundary of the bifocal diffractive structure and the outer refractive surface portion 19. By way of illustration, the step heights can be defined according to the following relation:

$$Step height = \frac{λ}{2 ( {n}_{2} - {n}_{1} )} {f}_{apodize} \qquad ( 3 )$$

wherein

λ, denotes the design wavelength (e.g., 550 nm),

n₂ denotes the refractive index of the material from which the lens is formed,

n₁ denotes the refractive index of a medium in which the lens is placed,

and f_{apodize} represents a scaling function whose value decreases as a function of increasing radial distance from the intersection of the optical axis with the anterior surface of the lens. For example, the scaling function can be defined by the following relation:

$${f}_{apodize} = 1 - {\{ \frac{( {r}_{i} - {r}_{in} )}{( {r}_{out} - {r}_{in} )} \}}^{exp} , {r}_{in} \leq {r}_{i} \leq {r}_{out} \qquad ( 4 )$$

wherein

rᵢ denotes a radial distance for the ith echelette defined as follows:
for i=0, a selected starting radius for the diffractive structure,
for i>0, rᵢ²=r₀²+2*iλf,

rᵢₙ denotes the inner boundary of the diffractive region as depicted schematically in FIG. 1A by the dashed line A,

rₒᵤₜ denotes the outer boundary of the diffractive region as depicted schematically in FIG. 1A by the dashed line B, and

exp is a value chosen based on the relative location of the apodization zone and a desired reduction in diffractive element step height. The exponent exp can be selected based on a desired degree of change in diffraction efficiency across the lens surface. For example, exp can take values in a range of about 2 to about 6.

As another example, the scaling function can be defined by the following relation:

$${f}_{apodize} = 1 - {( \frac{{r}_{i}}{{r}_{out}} )}^{3} \qquad ( 5 )$$

wherein

rᵢ denotes the radial distance of the i^{th} zone, and

rₒᵤₜ denotes the radius of the apodization zone.

Further details regarding selection of the step heights can be found in U.S. Pat. No. 5,699,142, which is herein incorporated by reference in its entirety.

The monofocal diffractive structure 18 of the IOL 10 exhibits a negative longitudinal chromatic aberration. That is, its optical power increases with increasing wavelength (its focal length decreases for longer wavelengths). In contrast, the refractive power provided by the IOL 10 as well as the human eye exhibit a positive chromatic aberration characterized by a decrease in optical power (increase in focal length) as a function of an increase in wavelength. Hence, the monofocal diffractive structure can be adapted to compensate for the positive chromatic aberration of the human eye and that of the lens itself for far and/or near vision. The negative chromatic aberration exhibited by the monofocal diffractive structure 18 can be adapted to counteract the positive chromatic aberration of the eye and that of the IOL itself so as to reduce the total chromatic aberration associated with the optical system comprising the IOL and the eye.

As noted above, the bifocal diffractive structure provides a far-focus optical power corresponding to its zero^{th} order diffraction, which in this case coincides substantially with the optical power of the monofocal diffractive structure and the refractive power of the lens, as well as a near-focus optical power corresponding to its 1^{st} order diffraction. Similar to the monofocal diffractive power, the near-focus optical power of the bifocal diffractive structure exhibits a negative chromatic aberration, which can at least partially compensate for the positive chromatic aberration of the eye (e.g., in the case of a phakic IOL that is implanted in an eye that retains its natural crystalline lens) for near vision. The above relation shows that the near-focus power of the bifocal structure is associated with a negative chromatic aberration, which can be adapted to counteract the positive chromatic aberration associated with the natural eye.

The above IOL 10 can advantageously provide improved distance vision due to chromatic aberration correction, e.g., for small pupil sizes in a range of about 2 mm to about 3 mm, a near optical power via the bifocal structures, e.g., for medium pupil sizes in a range of about 2.5 mm to about 3.5 mm, and good night vision.

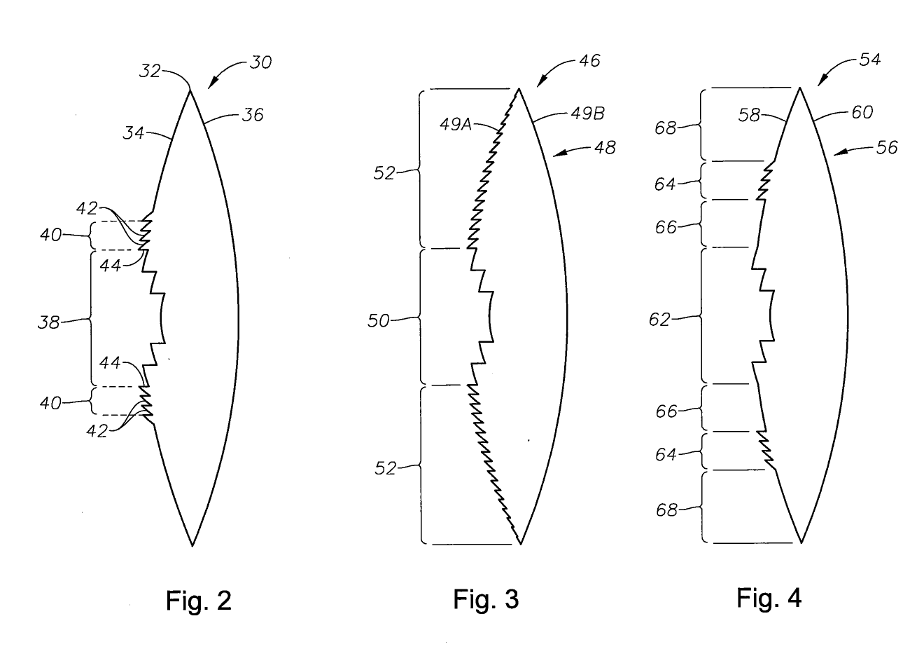

While in the above embodiment, the bifocal structure includes steps that exhibit a decreasing height as a function of increasing distance from the center of the anterior surface, in some other embodiments, the step heights separating the bifocal diffractive echelettes are substantially uniform. By way of example FIG. 2 schematically depicts such an IOL 30 that includes an optic 32 having an anterior surface 34 and a posterior surface 36. Similar to the previous embodiment, a monofocal diffractive structure 38 is disposed on a central region of the anterior surface 34, and is surrounded by a truncated bifocal structure 40. The bifocal structure 40 includes a plurality of diffractive echelettes 42 that are separated from one another by a plurality of steps. In this embodiment, the step height between adjacent echelettes of the bifocal structure, or the vertical height of each diffractive echelette at a zone boundary, is substantially uniform and can be defined according to the following relation:

$$Step height = \frac{b λ}{( {n}_{2} - {n}_{1} )} \qquad ( 6 )$$

wherein

λ denotes the design wavelength (e.g., 550 nm),

n₂ denotes the refractive index of the material from which the lens is formed,

n₁ denotes the refractive index of the medium in which the lens is placed, and

b is a fraction, e.g., 0.5 or 0.7.

Although in the above embodiments the bifocal diffractive structure is truncated, that is, it does not extend to the periphery of the lens, in other embodiments, the bifocal diffractive structure can extend to the lens's periphery. By way of example, FIG. 3 schematically depicts such a lens 46 that includes an optic 48 having an anterior surface 49A and a posterior surface 49B. Similar to the previous embodiments, a monofocal diffractive structure 50 is disposed on a central region of the anterior surface 49A, and is surrounded by a bifocal diffractive structure 52 that extends from the outer boundary of the monofocal structure to the periphery of the lens. The bifocal structure can include a plurality of diffractive echelettes that are separated from one another by a plurality of step heights, which can have a substantially uniform or apodized heights, e.g., in a manner discussed above. In this case, the step associated with the bifocal structure exhibit decreasing heights as a function of increasing distance from the center of the anterior surface.

FIG. 4 schematically depicts an IOL 54 according to another embodiment having an optic 56 with an anterior surface 58 and a posterior surface 60. A monofocal diffractive structure 62 is disposed on a central portion of the anterior surface. The anterior surface further includes a bifocal diffractive structure 64 that is separated from the monofocal diffractive structure 62 by an annular refractive region 66. An outer refractive region 68 surrounds the bifocal structure.

With continued reference to FIG. 4, in this example, the monofocal diffractive structure 62 provides a single diffractive focusing power corresponding to the IOL's far-focus power. The refractive regions 66 and 68 are configured, together with the refractive posterior surface 60, to provide a refractive optical power that is substantially equal to the far-focus power provided by the monofocal diffractive structure. The bifocal diffractive structure 64 in turn provides a far-focus power, which is substantially equal to the diffractive optical power provided by the monofocal diffractive lens and the refractive power provided by the refractive regions 66 and 68 in cooperation with the posterior surface. In addition, the bifocal diffractive structure 52 provides a near-focus optical power, e.g., a power in a range of about 1 to about 4 D. Although in this exemplary embodiment, the bifocal structure includes steps exhibiting apodized heights, in the other embodiments the respective step heights can be substantially uniform.

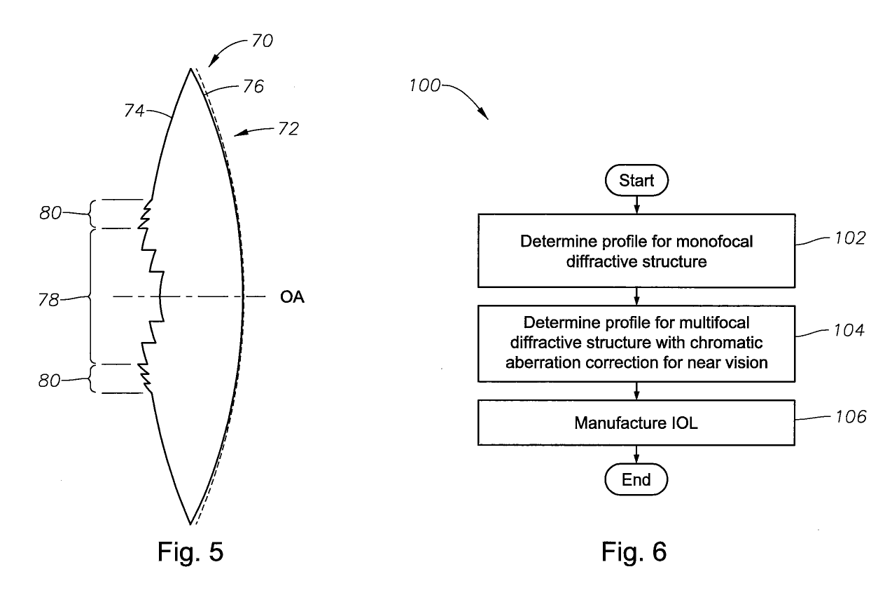

In some embodiments, a degree of asphericity can be imparted to the base profile of the anterior and/or the posterior surface of an IOL so as to ameliorate, and preferably eliminate, spherical aberrations effects. By way of example, FIG. 5 schematically depicts such an IOL 70 that includes an optic 72 having an anterior surface 74 and a posterior surface 76 disposed about an optical axis OA. Similar to the previous embodiments, a monofocal diffractive structure 78 is disposed on a central region of the anterior surface 74 while a bifocal diffractive structure 80 in the form of an annular region surrounds the monofocal diffractive structure. The base profile of the posterior surface deviates from a putative spherical profile (shown by dashed lines), with the deviation progressively increasing as a function of increasing distance from the center of the posterior surface defined in this case as the intersection of the optical axis with the posterior surface. In some embodiments, the asphericity of the base profile of the posterior surface can be characterized by a conic constant in a range of about −1030 to about −11. The asphericity can ameliorate, and preferably eliminate, spherical aberrations exhibited by the IOL. Although in this embodiment the base profile of the posterior surface is adapted to exhibit a degree of asphericity, in other embodiments, such an asphericity can be imparted to the anterior surface or both surfaces.

FIG. 6 is a flowchart 100 depicted an example method of manufacturing an IOL according to particular embodiments of the present invention. At step 102, a profile for a monofocal diffractive structure according to any of the various embodiments described herein with any suitable variations that would be apparent to one skilled in the art is determined. In particular, the determination of the monofocal diffractive profile can take into account desired power, suitable base curves for the anterior and/or posterior surfaces, asphericity or other aberration correction imparted to one or both surfaces, and the like. A focus of the monofocal diffractive structure can be selected, for example, to be a near-vision focus, a distance-vision focus, or an intermediate-vision focus. At step 104, a profile for a multifocal diffractive structure providing chromatic aberration correction for near vision is determined according to any of the various embodiments described herein with any suitable variations that would be apparent to one skilled in the art. In particular, the determination of the multifocal diffractive profile can take into account desired power, suitable base curves for the anterior and/or posterior surfaces, asphericity or other aberration correction imparted to one or both surfaces, and the like. In a particular example, the multifocal diffractive structure may be a bifocal diffractive structure with foci corresponding to a near-vision focus and a distance-vision focus. At step 106, an IOL with the monofocal diffractive structure and the multifocal diffractive structure having the respective profiles determined in steps 102 and 104 is manufactured. Suitable manufacturing techniques may include any method of formation suitable to the materials, including but not limited to molding, ablating and/or lathing.

Those having ordinary skill in the art will appreciate that various changes can be made to the above embodiments without departing from the scope of the invention. For example, rather than disposing both the monofocal and the multifocal diffractive structures on a single lens surface, one structure can be disposed on the lens's anterior surface and the other on its posterior surface. Further, the base profiles of the anterior and posterior surfaces can be configured such that the lens body would contribute refractively to the IOL's near-focus optical power.

1. An ophthalmic lens, comprising
an optic having an anterior surface and a posterior surface,
a monofocal diffractive structure disposed on one of said surfaces for providing a diffractive focusing power, and
at least one multifocal diffractive structure disposed on one of said surfaces for providing a plurality of diffractive focusing powers, wherein said multifocal diffractive structure is adapted to provide chromatic aberration compensation for near vision.

2. The ophthalmic lens of claim 1, wherein said monofocal diffractive structure provides a far-focus optical power.

3. The ophthalmic lens of claim 2, wherein said multifocal diffractive structure provides a near-focus optical power and a far-focus optical power.

4. The ophthalmic lens of claim 3, wherein the far-focus optical power provided by the monofocal diffractive structure is substantially equal to the far-focus optical power provided by the multifocal diffractive structure.

5. The ophthalmic lens of claim 1, wherein said monofocal diffractive structure is disposed on a central region of one of said surfaces.

6. The ophthalmic lens of claim 5, wherein said multifocal diffractive structure is disposed on an annular region of one of said surfaces surrounding said monofocal diffractive structure.

7. The ophthalmic lens of claim 5, wherein said anterior surface comprises an outer refractive region extending from an outer boundary of said annular region to a periphery of the lens.

8. The ophthalmic lens of claim 1, wherein said multifocal diffractive structure comprises a plurality of diffractive echelettes separated from one another by a plurality of steps.

9. The ophthalmic lens of claim 8, wherein said steps exhibit non-uniform step heights.

10. The ophthalmic lens of claim 8, wherein said non-uniform step heights are characterized by decreasing heights as a function of increasing distance from a center of the lens.

11. The ophthalmic lens of claim 1, wherein said lens comprises an IOL.

12. An ophthalmic lens, comprising
an optic having an anterior surface and a posterior surface,
a monofocal diffractive region disposed on a central portion of one of said surfaces, and
a bifocal diffractive annular region surrounding said monofocal diffractive region wherein said bifocal diffractive annular region is adapted to provide chromatic aberration compensation for near vision.

13. The ophthalmic lens of claim 12, wherein said monofocal diffractive region is adapted to provide a far-focus optical power.

14. The ophthalmic lens of claim 12, wherein said bifocal diffractive region is adapted to provide a far-focus and a near-focus optical power.

15. The ophthalmic lens of claim 12, wherein said lens comprises an IOL.

16. An ophthalmic lens, comprising
an optic having an anterior surface and a posterior surface,
a monofocal diffractive structure disposed on one of said surfaces to provide a far-focus optical power, said monofocal diffractive structure being adapted to provide compensation for chromatic aberration for far vision, and
a bifocal diffractive structure disposed on one of said surfaces so as to provide a far-focus optical power and a near-focus optical power, wherein said bifocal diffractive structure is adapted to provide chromatic aberration compensation for near vision.

17. The ophthalmic lens of claim 16, wherein at least one of said anterior or posterior surface exhibits an aspheric base profile.

18. A method of manufacturing an IOL, comprising:
determining a first surface profile for a monofocal diffractive structure disposed on either an anterior surface or a posterior surface of an IOL for providing a diffractive focusing power,
determining a second profile for at least one multifocal diffractive structure disposed on either the anterior surface or the posterior surface of the IOL for providing a plurality of diffractive focusing powers, wherein the multifocal diffractive structure is adapted to provide chromatic aberration compensation for near vision; and
manufacturing the IOL.

19. The method of claim 18, further comprising selecting said monofocal diffractive structure so as to provide a far-focus optical power.

20. The method of claim 18, further comprising selecting said multifocal diffractive structure to provide a far-focus and a near-focus optical power.

21. The method of claim 18, further comprising selecting said monofocal diffractive structure so as to provide chromatic aberration compensation for far vision.
